(19) United States
(12) Patent Application Publication
(10) Pub. No.: US 2006/0205073 A1
(43) Pub. Date: Sep. 14, 2006

(54) Aspirating oocytes from transplanted ovarian tissue

(75) Inventors: Kutluk H. Oktay, Rye, NY (US); Rodney W. Bosley, Chester Springs, PA (US); Jessica W. Miller, Terre Haute, IN (US)

Correspondence Address: BRINKS HOFER GILSON & LIONE, P.O. BOX 10395, CHICAGO, IL, 60610, US

(73) Assignee: Vance Products Incorporated, d/b/a Cook Urological Incorporated, Spencer, IN (US)

(21) Appl. No.: 11/370,545

(22) Filed: Mar. 8, 2006

Related U.S. Application Data
(60) Provisional application No. 60/659,503, filed on Mar. 8, 2005.

(51) Int. Cl. C12N 5/08; A61M 31/00

(52) U.S. Cl. 435/366; 604/500

(57) ABSTRACT

A method for aspirating an oocyte from a human female is provided. The method includes the step of providing at least one ovarian cortical piece, implanting the ovarian cortical piece in the human female at a heterotopic location, and triggering oocyte maturity in the at least one ovarian cortical piece. The method also includes the steps of providing an aspiration needle and retrieving at least one oocyte from the at least one ovarian cortical piece using the aspiration needle.

This application claims the benefit of the filing date under 35 U.S.C. § 119(e) of Provisional U.S. Patent Application Ser. No. 60/659,503, filed on Mar. 8, 2005, which is hereby incorporated by reference in its entirety.

### TECHNICAL FIELD

The present invention relates to a surgically-assisted reproduction procedure and device, and more particularly, to a method and apparatus for the aspiration of oocytes from transplanted ovarian tissue.

### BACKGROUND

Chemotherapy, radiotherapy, and radical surgery result in ovarian failure and infertility in hundreds of thousands of reproductive age women in the US alone. Thousands more may receive chemotherapy and radiation for the treatment of conditions such as collagen vascular, hematologic, and idiopathic diseases. Women of reproductive age with a partner may undergo an in vitro fertilization (“IVF”) cycle to cryopreserve their embryos prior to chemotherapy. IVF involves the fertilization of an oocyte or egg in vitro (outside of the womb). However, most cancer patients do not have enough time to complete the necessary ovarian stimulation for IVF. Also, this option is not acceptable for single women who do not wish to use donor sperm, or for children.

Oocyte cryopreservation is one experimental method that can be considered for adult women. Oocyte cryopreservation first requires oocyte retrieval. Typically, oocytes are retrieved from ovarian follicles (sac-like structures on the ovaries that contain the oocytes) by either a laparoscopic or transvaginal procedure using an aspiration needle. During an oocyte retrieval procedure, a relatively long aspiration needle is either vaginally or abdominally inserted into a patient so that the distal end of the needle is in contact with a patient's ovary. The objective is to puncture an individual follicle on the ovary and withdraw a single oocyte through the needle. A vacuum source, which is connected to the needle through flexible tubing, draws the ovary through the needle and tubing into a test tube.

Oocyte cryopreservation has its drawbacks, however. Pregnancy rates are generally low with this strategy, and as with embryo freezing, many cancer patients lack the several weeks necessary for ovarian stimulation before standard oocyte cryopreservation may be performed. Further, due to the large and complex nature of an oocyte, damage to the cell spindle, oocyte cytoskeleton, or zona pellucida (the membrane that surrounds the ovum) frequently occurs after cryopreservation and thawing. This results in a low pregnancy rate of less than three percent. Freezing the entire ovary is also not a viable alternative, as human ovaries are too large and fibrous to freeze in their entirety.

To deal with this, techniques have been developed where the ovarian cortex is cut into 1 to 2 mm-thick slices, each no larger than 0.5×1 cm dimensions. These slices are then placed in cryovials with a cryoprotectant, such as Leibovitz L-15 medium with 1.5 M 1,2-propanediol, 20% autologous serum, and 0.1 M sucrose, to penetrate the tissue. These cryovials are then frozen. After a patient completes her chemotherapy and bone marrow transplantation, these tissues are thawed and histologically tested to rule out any ovarian involvement with cancer. After experiencing some success in animal studies, human studies were conducted. In these studies, the thawed ovarian cortical pieces were implanted either orthotopically at the ovary sites or heterotopically in a forearm location back into the patient. However, while ovarian function was temporarily restored in the human studies and intact oocytes were retrieved, the oocytes did not become fertilized.

What is needed is a technique that allows for heterotopic transplantation of frozen thawed ovarian cortical pieces and subsequent retrieval of oocytes with a better IVF success rate than previous methods. Further, such a technique would utilize a new aspiration needle specifically adapted for such heterotopic sites, because existing aspiration needles are too long and unwieldy to accurately manipulate.

### BRIEF SUMMARY

Accordingly, embodiments of the present invention provide a new and improved method and apparatus for aspirating oocytes from transplanted ovarian tissue using an aspiration needle. In one embodiment, the ovarian tissue is transplanted to a subcutaneous heterotopic location. The needle is adapted with a suitable length to allow for aspiration at that heterotopic location.

According to a first aspect of the invention, a method for aspirating an oocyte for a human female is provided. The method includes the step of providing at least one ovarian cortical piece, implanting the ovarian cortical piece in the human female at a heterotopic location, and triggering oocyte maturity in the ovarian cortical piece. The method also includes the steps of providing an aspiration needle and retrieving at least one oocyte from the ovarian cortical piece using the aspiration needle.

According to a second aspect of the invention, a method for aspirating an oocyte from a human female is provided. The method includes providing at least one ovarian cortical piece, implanting the ovarian cortical piece in the human female at a heterotopic location, suppressing ovarian function in the human female, stimulating ovarian function in the human female, and triggering oocyte maturity in the ovarian cortical piece. The method also includes the steps of providing an aspiration needle and retrieving at least one oocyte from the ovarian cortical piece using the aspiration needle.

According to a third aspect of the invention, an aspiration needle is provided. The needle includes a cannula, an aspiration line, and a stopper. The cannula has an echogenic region and a beveled tip, wherein the cannula has a length of approximately 3 to 9 centimeters. The aspiration line has a first end and a second end, with the first end coupled to the cannula. The second end of the aspiration line extends through the stopper. The cannula and aspiration line are used to retrieve at least one oocyte from at least one ovarian cortical piece at a subcutaneous heterotopic location in a human female.

### BRIEF DESCRIPTION OF THE DRAWINGS

FIG. 1 is a flowchart for a method of embryo development after heterotopic transplantation of cryopreserved ovarian tissue using an aspiration needle;

FIG. 2 is a side view of the aspiration needle of the present invention;

FIG. 3 illustrates the aspiration needle of FIG. 2 inserted into a medical patient; and

FIG. 4 illustrates a detail view of the aspiration needle depicted in FIG. 3, inserted into a patient.

FIG. 5 is a graphical depiction of the resumption of ovarian function after transplantation.

FIG. 6 is a graphical depiction of the stimulation type, peak oestradiol concentration, and follicle sizes on day of human chorionic gonadotropin administration and oocyte retrieval outcome from ovarian transplant.

### DETAILED DESCRIPTION

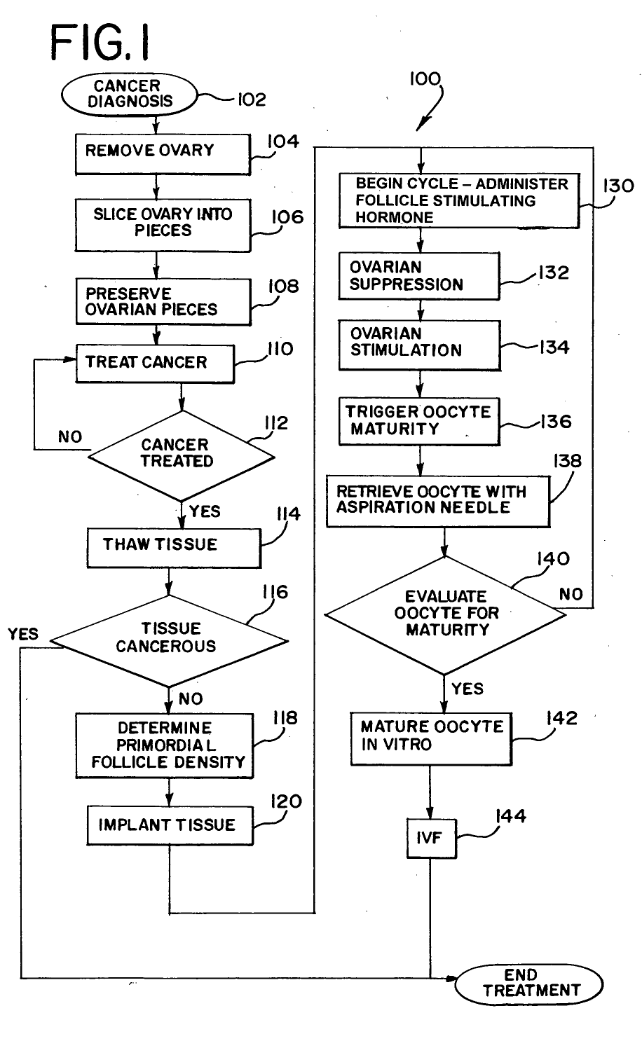

Referring now to FIG. 1, a method 100 for embryo development after heterotopic transplantation of cryopreserved ovarian tissue will be explained. A woman was first diagnosed with cancer 102 or a collagen vascular, hematologic, or idiopathic disease requiring chemotherapy, radiotherapy, or a radical surgery that results in ovarian failure and infertility. Ovarian tissue was collected via laparoscopy 104 or some other technique (such as transvaginal or abdominal surgery). In most cases, only one ovary will be removed, both to minimize trauma and because a single ovary provides a large amount of tissue. The ovary should be resected without the use electrosurgery, and the fallopian tube should be left intact so that the possibility of spontaneous pregnancy after future transplantation is maintained.

Once the ovary was removed, the ovarian cortex was sliced 106 into pieces ranging from 5×5×1 mm to 15×5×2 mm, although other sizes may also be used. These ovarian cortical pieces may be examined via biopsy prior to freezing to confirm that the ovarian tissue is free of any disease. These pieces were then cryopreserved 108 by placing the pieces into cryovials containing Leibovitz L-15 medium with 1.5 M 1,2-propanediol, 20% autologous serum, and 0.1 M sucrose. The cryovials were placed on a rotator and agitated for 30 minutes at 4° C. Then the ovarian pieces were frozen using a slow-freeze protocol, in a programmable freezer. One technique for harvesting, cryopreserving, thawing, and transplanting ovarian tissue is disclosed in the book, “A Color Atlas for Human Assisted Reproduction.” See chapter fourteen, entitled “The Technique of Ovarian Transplantation: Laboratory and Clinical Aspects” by Kutluk Oktay and Erkan Buyuk (“The Technique of Ovarian Transplantation”), which is herein incorporated by reference.

After the ovarian tissue was preserved cryogenically, the cancer, collagen vascular, hematologic, or idiopathic disease was treated with chemotherapy, radiotherapy, or radical surgery 110. Once successful treatment is verified 112, one vial of the ovarian cortical tissue was thawed 114. The tissue was histologically tested 116. The tissue was also tested to establish the density of primordial follicles 118. On the basis of primordial follicle density and the number of pieces available, it is possible to estimate how many follicles remain, and how long they could potentially last.

Under local anesthesia, the ovarian cortical pieces were implanted 120 in a heterotopic location beneath the skin of the patient's lower abdominal wall using a suture pull-through technique. First, a free end of a suture was threaded onto a half-circle cutting needle with a chord length ranging from 25 to 38 mm. Next, the needle was inserted into a subcutaneous pocket. The needle was then passed through the skin, and the cortical piece was wedged into the subcutaneous pocket by pulling on the suture. Additional details regarding the suture pull-through technique may be found in the Technique of Ovarian Transplantation, described above. The ovarian cortical pieces may alternately be implanted in other heterotopic locations, such as subcutaneously in the forearm. Although requiring IVF to fertilize any subsequently retrieved oocytes, a heterotopic site may be preferable to an orthotopic site because the technique does not require general anesthesia or abdominal surgery. Further, the ovarian tissue may be closely monitored. If there is a cancer recurrence or cyst formation, for example, ovarian removal is easily accomplished from heterotopic locations. However, oocyte quality may be affected because of differences in temperature and blood flow to a heterotopic subcutaneous environment compared with an orthotopic pelvic location.

Before every cycle of ovarian stimulation 132, ovarian suppression 130 was first performed by administering gonadotropin-releasing hormone antagonist or agonist (GnRH). Ovarian stimulation 134 was then performed with a combination of follicle stimulating hormone (FSH) and human menopausal gonadotropins (HMG). Oocyte maturity 136 was triggered by administering 250 μg of recombinant human chorionic gonadotropin (hCG), such as Ovidrel®, available from Serono, located in Norwell, Mass., USA.

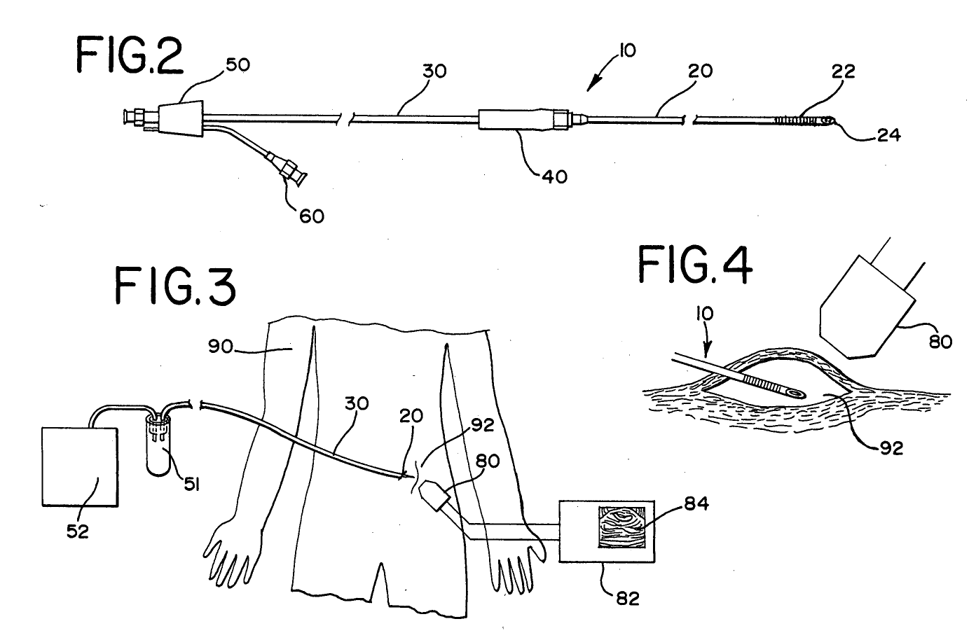

Oocyte retrieval 138 was performed thirty-six to forty hours after triggering oocyte maturity 136 using a modified aspiration needle 10, as seen in FIG. 2. As seen in FIGS. 3 and 4, an ultrasonic probe 80 may be used to provide imaging. Using this imaging technique, aspiration needle 10 may be used to pierce an individual follicle on an ovarian cortical piece and withdraw a single oocyte up through the aspiration needle 10. The aspiration needle 10, preferably with echogenic region 23 extending to the beveled tip 24, was inserted into a patient 90 at a heterotopic location beneath the skin of the patient's lower abdominal wall 92. Aspiration needle 10 was sonically imaged using an imaging device 82 with probe 80 to create image 84.

The aspiration needle 10 is made up of a needle cannula 20, a handle 40, an aspiration line 30, a bung or stopper 50, and a vacuum line 60. The needle cannula 20 has an echogenic region 22 with a beveled tip 24. Echogenic region 22 incorporates components that are similar in design and/or function as described in U.S. Pat. No. 5,081,997, issued Jan. 21, 1992 and entitled Echogenic Devices, Materials, and Method. The contents of this patent are hereby incorporated by reference to avoid unnecessary duplication of the description of similar components.

The aspiration needle 10 is modified from the Echotip® Ovum Aspiration Needle, available from COOK®, Cook Urological Inc., Spencer, Ind., USA. However, due to the heterotopic location of the implanted ovarian cortical pieces and the smaller volumes of the immature follicles, the needle cannula 20 and aspiration line 30 are reduced in size. The needle cannula 20 is preferably made from 19 gauge 304 stainless steel thinwall tubing, with an overall length of 4 cm, an outer diameter of 0.042 inches, and an inner diameter of 0.032 inches. However, needle lengths from 3 cm to 9 cm may also be used. In addition, other biocompatible materials, such as polycarbonates, may be used. Moreover, other tubing gauges or thicknesses may also be used. Preferably, the tubing is small enough to allow for accurate needle placement and oocyte retrieval, yet large enough to avoid needle flexibility. For example, tubing from 17 to 20 gauge may be used. The aspiration line 30 is preferably made from a 30 cm length of translucent 19 gauge TFE (TEFLON™) tubing, although other biocompatible materials and sizes may also be used. For example, aspiration line 30 may have a length from 15 to 60 cm, and a tubing gauge sized to match needle cannula 20.

Handle 40 couples needle cannula 20 with one end of aspiration line 30. Handle 40 also provides a gripping surface to precisely manipulate aspiration needle 10. The other end of aspiration line 30 passes through stopper 50 and terminates in a luer lock connection. Stopper 50 is a formed as a silicone plug and is adapted to provide an air-tight seal for a collection tube 51. Vacuum line 60 also extends through stopper 50. A vacuum source 52 may be attached to a luer lock connection on vacuum line 60. Vacuum source 52 provides a means for withdrawing an oocyte from a patient's follicle and depositing the oocyte in the collection tube 51 attached to stopper 50. Due to the reduced size of aspiration needle 10, a smaller vacuum pressure is needed than that used for standard needles designed for orthotopic aspiration. For example, a volume flow rate of 10-30 mL/minute with a vacuum pressure of 60-80 mm Hg may be used for aspiration needle 10. In contrast, for standard aspiration needles, volume flow rates of 30-40 mL/minute with vacuum pressures of 110-130 mm Hg may be required.

Progesterone supplements were administered during every cycle, following oocyte retrieval. After oocytes were retrieved using aspiration needle 10, they were evaluated to determine their maturity 140. Immature oocytes may be matured in vitro 142. The oocyte was matured and fertilized in a complex, non-commercial sequential culture medium. One hypothesis predicts that aspiration of immature oocytes from smaller follicles followed by in vitro maturation may be the preferred approach to preserve the competence to undergo fertilization. Further, oocyte maturity at heterotopic locations appears to be attained at 10-11 mm diameter, in contrast with a 16-17 mm diameter in orthotopic ovaries.

After the oocyte is matured, an IVF technique 144 such as intracytoplasmic sperm injection technique (ICSI) may be used to fertilize the oocyte, which will develop into an embryo. The oocyte and embryo was matured and cultured in this medium until about 48 hours after the injection. The embryo may then be evaluated to determine morphology, such as by a preimplantation genetic diagnosis by fluorescence and in-situ hybridization. Normal embryos are then transferred to a patient's uterus.

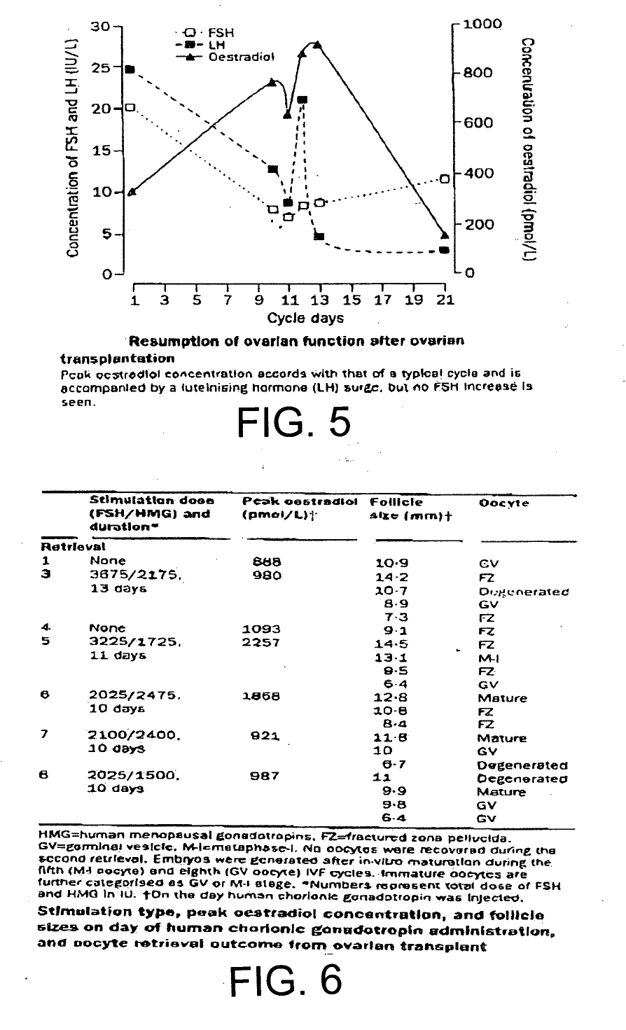

FIG. 5 illustrates the patient's resumption of ovarian function afer ovarian transplantation. The peak oestradiol concentration accords with that of a typical cycle and is accompanied by a luteinising hormone (LH) surge, but no FSH increase is seen.

The results of the patient's percutaneous oocyte retrievals are shown in FIG. 6. FIG. 6 is a graphical depiction of the stimulation type, peak oestradiol concentration, and follicle sizes on day of human chorionic gonadotropin administration and oocyte retrieval outcome from ovarian transplant. Eight consecutive cycles of retrievals were conducted over eight months. Of the approximately 20 oocytes obtained from the patient's transplanted tissue, eight were suitable for IVF with her husband's sperm. Three of the eight oocytes were mature at retrieval, while the other five oocytes had to be matured in vitro. Although the mature oocytes did not fertilize, two of the oocytes that were matured in vitro were fertilized via ICSI. One embryo showed abnormal morphology and its growth was halted at the three-cell stage. The other embryo was transferred to the patient's uterus.

While the invention has been described with reference to details of the illustrated embodiment, these details are not intended to limit the scope of the invention as defined in the appended claims.

1. A method for aspirating an oocyte from a human female comprising the steps of:
a. providing at least one ovarian cortical piece;
b. implanting the at least one ovarian cortical piece in the human female at a heterotopic location;
c. triggering oocyte maturity in the at least one ovarian cortical piece;
d. providing an aspiration needle; and
e. retrieving at least one oocyte from the at least one ovarian cortical piece using the aspiration needle.

2. The method of claim 1, wherein the aspiration needle further comprises a needle cannula with an echogenic region and a beveled tip, and an aspiration line coupled with the needle cannula.

3. The method of claim 2, wherein the needle cannula has a length of approximately 4 cm.

4. The method of claim 2, wherein the aspiration line has a length of approximately 30 cm.

5. The method of claim 1, wherein the heterotopic location is subcutaneous either in the forearm or the lower abdomen of the human female.

6. The method of claim 5, wherein the at least one ovarian cortical piece is implanted in the human female using a suture pull-through technique.

7. A method for aspirating an oocyte from a human female comprising the steps of:
a. providing at least one ovarian cortical piece;
b. implanting the at least one ovarian cortical piece in the human female at a heterotopic location;
c. suppressing ovarian function in the human female;
d. stimulating ovarian function in the human female;
e. triggering oocyte maturity in the at least one ovarian cortical piece;
f. providing an aspiration needle; and
g. retrieving at least one oocyte from the at least one ovarian cortical piece using the aspiration needle.

8. The method of claim 7, wherein the aspiration needle further comprises a needle cannula with an echogenic region and a beveled tip, and an aspiration line coupled with the needle cannula.

9. The method of claim 8, wherein the needle cannula has a length of approximately 4 cm.

10. The method of claim 8, wherein the aspiration line has a length of approximately 30 cm.

11. The method of claim 7, wherein the heterotopic location is subcutaneous either in the forearm or the lower abdomen of the human female.

12. The method of claim 7, wherein the at least one ovarian cortical piece is implanted in the human female using a suture pull-through technique.

13. The method of claim 7 further comprising the step of:
h. maturing the at least one oocyte by in vitro maturation.

14. An aspiration needle comprising:
a cannula with an echogenic region and a beveled tip, wherein the cannula has a length of approximately 3 to 9 centimeters;
an aspiration line having a first end and a second end, wherein the first end is coupled with the cannula; and
a stopper, wherein the second end of the aspiration line extends through the stopper; and
wherein the cannula and aspiration line are configured for retrieving at least one oocyte from at least one ovarian cortical piece at a subcutaneous heterotopic location in a human female.

15. The aspiration needle of claim 14, wherein the cannula has a length of approximately 4 centimeters.

16. The aspiration needle of claim 15, wherein the cannula has a tubing gauge from 17 to 21.

17. The aspiration needle of claim 16, wherein the aspiration line has a length of approximately 15 to 60 centimeters.

18. The aspiration needle of claim 14, wherein the heterotopic location is subcutaneous either in the forearm or the lower abdomen of the human female.

19. The aspiration needle of claim 14, wherein the echogenic region extends to the beveled tip.

20. The method of claim 1, wherein the aspiration needle is from 17 to 21 gauge.
